(19) United States
(12) Patent Application Publication
(10) Pub. No.: US 2004/0191218 A1
(43) Pub. Date: Sep. 30, 2004

(54) Pharmaceutical compositions and methods for treating cancer

(76) Inventors: James W Emlen, Greenwood Village, CO (US); Brent F G Treiger, Los Altos, CA (US)

Correspondence Address: Cooley Godward, Five Palo Alto Square, 3000 El Camino Real, Palo Alto, CA, 94306-2155, US

(21) Appl. No.: 10/490,791

(22) PCT Filed: Sep. 26, 2002
(86) PCT No.: PCT/US02/30621

(30) Foreign Application Priority Data
Sep. 26, 2001 (US) 60325246

(51) Int. Cl. A61K 38/21; A61K 33/24; A61K 31/337

(52) U.S. Cl. 424/85.5; 424/85.6; 424/649; 514/449

(57) ABSTRACT

A method is provided for treatment of cancer, particularly ovarian cancer, using a combination of a taxane, a platinum coordination compound and interferon-gamma. In particular, the method provides treatment of ovarian cancer with a combination of paclitaxel, carboplatin and interferon-gamma. The addition of interferon-gamma to the standard chemotherapeutic treatment using paclitaxel and carboplatin provides an improved therapeutic outcome.

### INTRODUCTION

1. Technical Field

This invention relates to a combination therapy of a platinum coordination compound, a taxane, and interferon gamma (IFN-γ) to treat various cancers, including ovarian cancer.

2. Background

In the United States, ovarian cancer is the leading cause of death from gynecologic malignancies and is fourth overall in women behind lung, breast and colorectal cancer. In the year 2000, it was estimated that there were 23,100 new cases diagnosed and 14,000 women died of this disease (Greenlee et al., Cancer Statistics, 2000, CA Cancer J Clin 50 (1): 7-33, (2000)). In the past decade, the number of ovarian cancers has increased 30% and the number of ovarian cancer deaths has increased 18% (Wingo et al., Cancer Statistics, 1995, CA Cancer J Clin 8-30, (1995)).

Despite recent improvements in the treatment of ovarian cancer, the overall longterm survival has not changed significantly since 1960 (Greenlee et al., Cancer Statistics, 2000, CA Cancer J Clin 50 (1): 7-33, (2000)). Over 75% of ovarian cancer patients have advanced stage disease at the time of diagnosis (Greenlee et al., Cancer Statistics, 2000, CA Cancer J Clin 50 (1): 7-33, (2000)). Although a temporary response rate to chemotherapy of 70% can be anticipated, ovarian cancer tends to recur, even in patients who achieve a complete response, and the 5-year survival for patients with advanced ovarian cancer is less than 30% (Society of Gynecologic Oncologists Clinical Practice Guidelines. Practice Guidelines: Ovarian Cancer. Oncology 12(1): 129-133, (1998)).

The standard of care of patients with advanced ovarian cancer has changed several times over the past decade. In the early 1990s, the standard was the combination of a platinum compound (cisplatin or carboplatin) and an alkylating agent (i.e., cyclophosphamide). Then in the mid 1990s, two randomized Phase III trials showed a significant outcome advantage in patients with advanced ovarian cancer when cisplatin and paclitaxel were combined compared with cisplatin plus cyclophosphamide (McGuire et al, Cyclophosphamide and Cisplatin Compared with Paclitaxel and Cisplatin in Patients with Stage III and Stage IV Ovarian Cancer, NEJM 334 (1): 1-6, (1996); Piccart et al, Randomized Intergroup Trial of Cisplatin-Paclitaxel Versus Cisplatin-Cyclophosphamide in Women with Advanced Epithelial Ovarian Cancer: Three-Year Results, J Natl Cancer Inst 92 (9): 699-708, (2000)).

Carboplatin is often substituted for cisplatin because carboplatin is associated with less neurotoxicity and nephrotoxicity than cisplatin. An additional advantage of carboplatin is that it can be administered as an outpatient infusion and does not require intravenous hydration. Currently, treatment with a combination of carboplatin and paclitaxel has been adopted by most physicians in the U.S. as the standard of care and is used in more than 80% of advanced ovarian cancer patients as initial chemotherapy (McGuire, W P. Epithelial Ovarian Cancer. ASCO 2000 Educational Book 541-546, (2000)).

IFN-γ, which is a protein produced by activated T-cells and natural killer (NK) cells, has pleiotropic immunologic effects. In addition, IFN-γ exerts antiproliferative effects on neoplastic cells including ovarian cancer cells (Saito et al., Direct and Indirect Effects of Human Recombinant γ-Interferon on Tumor Cells in a Clonogenic Assay, Cancer Research 46: 1142-1147, (1986)). Clinical evidence for activity of IFN-γ for the treatment of ovarian cancer was suggested in 1998 by Welander and associates who treated 14 patients with relapsing ovarian cancer with IFN-γ (2 mg/m²) daily intravenously (Welander et al., A Phase 11 Study of the Efficacy of Recombinant Interferon Gamma in Relapsing Ovarian Adenocarcinoma, Am J Clin Oncol (CCT) 11 (4): 465-469, (1988)). Positive responses were observed in four patients (29%). Several clinical trials have been conducted with intraperitoneal IFN-γ for the treatment of ovarian cancer. While early trials in limited numbers of patients yielded contradictory results, Pujade-Lauraine et al. (Intraperitoneal recombinant interferon gamma in ovarian cancer patients with residual disease at second look laparotomy, J Clin Oncol 14: 343-350, (1996)) reported an overall response rate of 31% in a series of 108 patients with residual disease at second-look laparotomy. In vitro data further demonstrate synergistic inhibition of proliferation of ovarian cancer cells treated simultaneously with cisplatin and IFN-γ (Nehme et al., Modulation of Cisplatin Cytotoxicity by Human Recombinant Interferon-γ in Human Ovarian Cancer Cell Lines, Eur J Cancer 30A (4): 520-525, (1994)).

Windbichler and associates have evaluated the combination of IFN-γ1b and cisplatin based chemotherapy for first-line therapy of ovarian cancer (Windbichler et al, Interferon-gamma in the first-line therapy of ovarian cancer: a randomized phase III trial, British Journal of Cancer 82 (6): 1138-1144, (2000)). In this trial, chemotherapy consisted of cisplatin and cyclophosphamide which was the standard of care at the time the trial was started in 1991. Patients randomized to chemotherapy and interferon γ-1b received the same chemotherapy regimen plus IFN-γ1b (0.1 mg s.c. three times a week every other week). Median follow-up was 24 months and 29 months for progression-free survival (PFS) and overall survival (OS), respectively. PFS at 3 years was improved from 38% in controls to 51% in the treatment group corresponding to median times to progression of 17 and 48 months (P=0.031, relative risk of progression 0.48, confidence interval 0.28-0.82). Three-year overall survival was 58% and 74% accordingly (n.s., median not yet reached). Toxicity was comparable in both groups except for a mild flu-like syndrome experienced by many patients after administration of IFN-γ1b.

The current standard post-operative therapy for advanced ovarian cancer includes platinum based chemotherapy in combination with paclitaxel, in place of cyclophosphamide (Partridge and Barnes CA Cancer J Clin. 1999 49:297). Despite advances in chemotherapy, ovarian cancer continues to be fatal in far too many cases. New, more effective therapies are still needed.

### SUMMARY OF THE INVENTION

The present invention provides a method of treatment of cancer by administering a combination of interferon-gamma (“IFN-γ”) with a taxane and a platinum coordination compound. The method is particularly suited for treatment of ovarian cancer, primary peritoneal cancer, breast cancer, cervical cancer, small cell lung cancer, non-small cell lung cancer and cancers of the head and neck. Treatment of ovarian cancer is particularly preferred. For use in the method of the present invention, the preferred taxanes include paclitaxel, or taxol; preferred platinum coordination compounds include carboplatin and cisplatin; preferred forms of interferon-gamma include recombinant interferon-gamma, particularly interferon-gamma 1b (“IFN-γ1b”). The present invention provides an improvement in the current standard of care for ovarian cancer, the improvement comprising the administration of interferon-gamma, particularly interferon-gamma 1b, to patients undergoing chemotherapeutic treatment for ovarian cancer. The method of the present invention provides particular regimes for administration of IFN-γ in combination with a taxane and a platinum coordination compound (“Pt compound”) that achieve superior therapeutic results compared to those obtained by chemotherapy with a combination of a taxane and a Pt compound alone. In particular, the method of the present invention comprises administering an IFN-γ at a dose of at least about 0.1 mg, at a frequency of at least about three times a week, every week during the chemotherapy and for at least about three weeks following the final chemotherapy treatment. The IFN-γ is preferably IFN-γ 1b. A kit comprising interferon-gamma 1b packaged for use in the method of the present invention, together with instructions for use in the method is also provided.

### DESCRIPTION OF SPECIFIC EMBODIMENTS

This invention provides advantageous combination therapies for cancers, including without limitation, ovarian cancers, breast cancers, cervical cancers, peritoneal cancers, small cell lung cancers, non-small cell lung cancers and cancers of the head and neck. Treatment of ovarian cancer is particularly preferred. The present invention provides a method for treating cancer using a combination of a platinum coordination compound, a taxane, and interferon-gamma (IFN-γ). This combination provides an improved treatment method for ovarian cancer patients over current chemotherapeutic treatment methods that use the combination of a platinum coordination compound and a taxane. In a particular aspect the present invention provides an improved method of treatment for ovarian cancer patients which combines the administration of interferon-gamma with the presently used chemotherapeutic treatment with a taxane, selected from the group consisting of carboplatin and cisplatin, and paclitaxel. Kits comprising interferon-gamma in a form suitable for administration in the method of the present invention together with instructions for its use in the method of the present invention are an additional aspect of the invention. The kits may additionally comprise a taxane or a platinum coordination compound, or both, supplied in forms suitable for administration in the method of the present invention.

The present invention thus provides a method of treating cancer in a patient afflicted therewith which comprises administering to such patient an effective amount of a platinum coordination compound, a taxane, and IFN-γ. The use of IFN-γ together with a taxane and a platinum coordination compound for treatment of cancer represents an improvement over the treatment with a taxane and platinum coordination compound alone. The IFN-γ is administered in accordance with the therapeutic regimen described herein to achieve superior therapeutic results. In particular aspect, the present invention provides a method of treating ovarian cancer in a patient in need of such treatment comprising administering an effective amount of interferon-gamma during the course of chemotherapy with a taxane and a platinum coordination compound. In the method of the present invention, the IFN-γ is administered to patients undergoing conventional chemotherapy with a combination of a taxane and a Pt compound. The IFN-γ is administered at least about three times per week, every week for the duration of the chemotherapy and for at least about three weeks thereafter. The IFN-γ is administered in a dose of at least about 0.1 mg, three times per week to every other day. The preferred mode of administration is subcutaneous injection. For particular patients and/or particular cancers, the addition of interferon-gamma to the current standard chemotherapy will provide a synergistic effect compared to treatment with either interferon-gamma or chemotherapy alone.

The terms “interferon gamma”, “gamma-interferon” and “IFN-γ” are used interchangeably in this application in accordance with the interferon nomenclature announced in Nature, 286, p. 110 (1980) and recommended by the Interferon Nomenclature Committee in Archives of Virology, 77, pp. 283-85 (1983). IFN-γ was originally referred to as “immune interferon”.

IFN-γ is a lymphokine which is naturally produced in minute quantities together with other lymphokines by lymphocytes. It is primarily produced by T-lymphocytes, spontaneously or in response to various inducers such as mitogens, specific antigens or specific antibodies (W. E. Stewart, II, The Interferon System, pp. 148-49 (1981)). In its native form, IFN-γ is a glycoprotein having a molecular weight between 20,000 and 25,000 (or 17,000 in non-glycosylated form). The IFN-γ gene has also been cloned and expressed in various host-vector systems (Gray and Goeddel, Nature, 1982 298:859; Gray and Goeddel, Basic Life Sci, 1983 25:35; U.S. Pat. Nos. 4,762,791, 5,582,824, 4,855,238 and 5,595,888).

As used in this application, “IFN-γ” includes all proteins, polypeptides and peptides which are natural or recombinant IFN-γs, or derivatives thereof, and which are characterized by the biological activity of those IFN-γs against malignant, non-malignant or viral diseases. These include IFN-γ-like compounds from a variety of sources such as natural IFN-γs, recombinant IFN-γs, and synthetic or semi-synthetic IFN-γs.

Interferon gamma-1b (IFN-γ-1b) is a preferred form of IFN-γ in the present invention. IFN-γ-1b is well known and is described in U.S. Pat. No. 5,690,925, inter alia. IFN-γ-1b is commercially available, for example, under the trade name Actimmune®. For use in the present invention, the IFN-γ will typically be formulated in 20 mg mannitol, 0.36 mg sodium succinate, and 0.05 mg polysorbate 20, per 0.100 mg (2 million IU) of IFN-γ in Sterile Water for Injection. The IFN-γ can be administered in an amount from at least about 0.1 mg to at least about 10 mg, and includes at least about 0.15 mg, at least about 0.20 mg, at least about 0.25 mg, at least about 0.50 mg, at least about 0.75 mg, at least about 1.0 mg, at least about 1.5 mg, at least about 2.0 mg, at least about 3.0 mg, at least about 4.0 mg, at least about 5.0 mg, at least about 6.0 mg, at least about 7.0 mg, at least about 8.0 mg, at least about 9.0 mg, and at least about 10.0 mg. Preferably, the dosage will range from at least about 0.1 mg to at least about 1 mg, more preferably from at least about 0.1 mg to at least about 0.2 mg, with about 0.1 mg being the most preferred dose. The IFN-γ will be administered concurrently with the chemotherapeutic treatment and will continue to be administered after completion of the chemotherapy course. Generally, administration of IFN-γ will begin on the same day as the taxane/Pt compound chemotherapy. Typically, the IFN-γ will be administered following the administration of the taxane and Pt compound. The IFN-γ is administered at a frequency of at least about three times a week at regular intervals (for example, about every other day or Monday, Wednesday and Friday), to at least about every day. The interval between administration of IFN-γ can be adjusted to minimize any adverse side effects of the IFN-γ. Administration of the IFN-γ is continued after the completion of the chemotherapy for at least about three weeks following the final administration of chemotherapy. The preferred dosing schedule is IFN-γ 0.1 mg, subcutaneously, 3 times per week (every other day, for example, Monday/Wednesday/Friday) continuously while the patients are receiving chemotherapy with the taxane-Pt compound combination, and for 3 weeks following the last dose of chemotherapy.

Patients to be treated with the combination therapy provided here are those that have been diagnosed with ovarian cancer, including newly diagnosed and relapsing cases and metastatic ovarian cancer. Other patients that will benefit from the treatment methods of the present invention include patients who have been diagnosed with any cancer for which treatment with a taxane in combination with a platinum coordination compound is indicated. The method of the present invention is particularly beneficial for patients who have been diagnosed with any cancer for which treatment with paclitaxel in combination with carboplatin or paclitaxel in combination with cisplatin is indicated. Such suitable patients include those diagnosed with breast cancers, including metastatic breast cancer, cervical cancers, including advanced or recurrent cervical cancers, peritoneal cancers, small cell lung cancers, non-small cell lung cancers and cancers of the head and neck, including recurrent squamous cell carcinomas.

By the term “platinum coordination compound” is meant any tumor cell growth inhibiting platinum coordination compound which provides the platinum in the form of an ion. Such compounds are well known and many are commercially available. Preferred platinum coordination compounds include cis-diaminedichloroplatinum (Cisplatin); cis-diamminediaquoplatinum (II)-ion; chloro(diethylenetriamine)-platinum(II) chloride; dichloro(ethylenediamine)-platinum(II); diammine(1,1-cyclobutanedicarboxylato) platinum(II) (Carboplatin); Spiroplatin; Iproplatin; diammine(2-ethylmalonato)-platinum(II); ethylenediaminemalonatoplatinum(II); aqua(1,2-diaminodyclohexane)-sulfatoplatinum(II); (1,2-diaminocyclohexane)malonatoplatinum(II); (4-carboxyphthalato) (1,2-diaminocyclohexane)platinum(II); (1,2-diaminocyclohexane)-(isocitrato)platinum(II); (1,2-diaminocyclohexane)cis(pyruvato)platinum(II); and (1,2-diaminocyclohexane)oxalatoplatinum(II); Ormaplatin; Oxaloplatin; and Tetraplatin.

In the methods and compositions of the present invention, carboplatin and cisplatin are the preferred platinum coordination compounds. Carboplatin is particularly preferred. By the term “carboplatin” is meant diammine(1,1-cyclobutanedicarboxylato) platinum(II). Carboplatin is commercially available from Bristol Myers Squibb as Paraplatin.® Cisplatin (cis-diaminedichloroplatinum) is also available commercially from Bristol Myers Squibb as Platinol®. Other platinum coordination compounds named herein are known and are available commercially and/or can be prepared by conventional techniques. See U.S. Pat. Nos. 4,140,707 and 4,657,927.

In the method of the present invention, the platinum coordination compound will be administered in a manner found appropriate by a clinician in generally efficacious doses, for example, in an amount as recommended by the manufacturer for the treatment of the particular cancer to be treated. Suitable dosage information can be found in the Physicians' Desk Reference, 53Ed. 1999, Medical Economics Co. Typically, the dose of platinum coordination compounds, such as cisplatin or carboplatin, will be calculated to reach a target area under the curve (AUC) of concentration X time according to the Calvert formula using an estimated glomerular filtration rate (GFR) from the Jelliffe formula. This method of calculating dose is conventional and would be well within the competence of a medical practicioner of ordinary skill. For purposes of the present invention the dose of platinum coordination compound may be calculated as follows: Dose (mg)=target AUC X (GRF+25). Target AUC will range from AUC of 4 to AUC of 7, preferably AUC of 5 to AUC of 7, more preferably an AUC of 6 mg/mL×min. For most purposes, GRF can be considered as equivalent to creatinine clearance. Creatinine clearance (CCr) can be estimated by the method of Jelliffe using the formula: CCr=0.9 X {98-[0.8(age-20)]}/Scr, where CCr is the estimated creatinine clearance rate in ml/min, Age is the patient's age in years and Scr is the serum creatinine in mg/dl.

Alternatively, the dosage for platinum coordination compound, particularly cisplatin, will range from 50 to 100 mg/m² i.v.

The platinum coordination compound is generally administered as an i.v. bolus or rapid infusion over a period of approximately 1 hour but may be administered in any other appropriate manner. The manner of administration of the platinum compound is well within the competence of the medical practicioner to determine.

By the term “taxane” is meant any member of the family of terpenes, including, but not limited to paclitaxel and docetaxel (Taxotere), which were derived primarily from the Pacific yew tree, Taxus brevifolia, and which have activity against certain tumors, particularly breast and ovarian tumors (See, for example, Pazdur et al. Cancer Treat Res. 1993 19:351; Bissery et al. Cancer Res. 1991 51:4845). In the methods and compositions of the present invention, preferred taxanes are paclitaxel, docetaxel, and deoxygenated paclitaxel. Paclitaxel is particularly preferred. Without limitation to any particular theory, paclitaxel is thought to be an antimicrotubule agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions. The term “paclitaxel” includes both naturally derived and related forms and chemically synthesized compounds or derivatives thereof with antineoplastic properties including deoxygenated paclitaxel compounds such as those described in U.S. Pat. No. 5,440,056, U.S. Pat. No. 4,942,184, which are herein incorporated by reference, and that sold as TAXOL® by Bristol-Myers Oncology. Chemical formulas for paclitaxel are known and can be found in the two previous cited references or references cited therein. For example, in addition to TAXOL®, other derivatives are mentioned in “Synthesis and Anticancer Activity of Taxol other Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elvesier, Amsterdam 1986), pp 219-235 are explicitly included here.

The taxane may be administered in a manner found appropriate by a clinician in generally accepted efficacious dose ranges such as those described in the Physician Desk Reference, 53^{rd} Ed. (1999), Publisher Medical Economics Co., New Jersey (“PDR”) for paclitaxel. Regimes for administration of paclitaxel or taxol are described, inter alia, in U.S. Pat. No, 5,641,803. In general, the taxane is administered intravenously at dosages from about 135 to about 300 mg/m², preferably from about 135 to about 175 mg/m², and most preferably about 175 mg/m². It is preferred that the dosages be administered over a time period of about 1 to about 24 hours, typically over a period of about 3 hours. For the method of the present invention, the taxane dosages can be repeated from about every 3 days to about every 28 days, preferably from about every 14 days to about every 21 days, more preferably every 21 days (i.e., three weeks), for a total period of at least about 15 weeks.

Provided other formulations of paclitaxel may be tolerated by a patient, the drug may be administered in any other form such as by injection or oral forms. Liposome formulations, for example, have been described. See, e.g. U.S. Pat. No. 5,424,073, which is herein incorporated by reference.

In the practice of the method of the present invention, the taxane and the platinum coordination compound are typically administered sequentially, generally on the same day. They may be administered in any order, although it is preferable that the taxane is administered first over a period of from 3 to 24 hours, typically from 3 to 4 hours, followed by the platinum compound, which is typically administered over a period of no more than 1 hour. Particularly for the treatment of ovarian cancer, the administration of a taxane, eg, paclitaxel, followed by a Pt compound, eg, carboplatin or cisplatin, is well known and the protocols and regimens for use of these compounds in combination are conventional and well within the competence of the medical practicioner of ordinary skill to determine (see, DeVita et al. Eds Cancer Principles and Practice of Oncology 5^{th} Ed. 1997 Lippincott-Raven Philadelphia). Typically, the taxane-Pt compound therapy is administered once every three weeks for 6 rounds of therapy. A particularly preferred chemotherapeutic treatment is one wherein paclitaxel is administered intravenously at a dose of from about 135 mg/m² to about 175 mg/m² and carboplatin is administered intravenously at an AUC of 5 to an AUC of 7.

The term “effective amount” as used herein is meant a course of therapy which will result in a beneficial outcome for cancer treatment. The effective amount when referring to the taxane or the Pt compound, or the combination thereof, will generally mean the dose range(s), modes of administration, formulations, etc., that have been recommended or approved by any of the various regulatory or advisory organizations in the medical or pharmaceutical arts (eg, FDA, AMA) or by the manufacturer or supplier. Effective amounts for the taxane and the Pt compound can be found, for example, in the Physicians' Desk Reference (supra). When referring to the IFN-γ, the effective amount is the dose range and administration regimen described herein. It will be appreciated that the actual preferred course of therapy will vary according to, inter alia, the mode of administration, the particular formulation of compounds being utilized, and the particular patient being treated. The optimal course of therapy for a given set of conditions can be ascertained by those skilled in the art using conventional course of therapy determination tests in view of the information set out herein.

By the term “ovarian cancer” as used herein is meant adenocarcinoma of the ovary and includes primary peritoneal cancers. By the term “treating ovarian cancer” as used herein is meant inhibiting of the growth of ovarian cancer cells, prolonging the periods of disease remission, increasing the progression-free survival time, overall survival or other measure that is conventionally used to determine beneficial therapy. Preferably such treatment also leads to the regression of tumor growth, i.e., the decrease in size of a measurable tumor. Most preferably, such treatment leads to the complete regression of the tumor. Typically, the efficacy of the treatment method is determined by measuring the percentage of treated patients exhibiting progression-free survival (“PFS”) three years after completion of the treatment, but PFS data obtained at earlier time points (for example, 1 year or 18 months after treatment completion) can provide meaningful information. The improvement provided by the method of treatment of the present invention can be determined by measuring the percentage of treated patients exhibiting PFS three years after completion of treatment according to the present invention compared to the percentage of patients exhibiting PFS three years after chemotherapy with a combination of taxane and Pt compound alone. PFS can also be measured at longer times after treatment completion, for example, four years or five years out. For determination of PFS, patient populations should be sufficiently large to provide statistically meaningful data and the patient population are randomized for other factors as is well known in the art. By “progression free survival” is intended survival of the patient in the absence of progression of the disease as measured by any one of the criteria for measuring progression. Progression of the disease is determined by the presence of any one of the following:

Greater than or equal to a 20% increase in the sum of the longest diameters of target lesions, taking as reference the smallest sum of the longest diameters recorded since the treatment started.

Greater than or equal to a 20% increase in the diameter of any lesion (target or non-target), taking as reference the smallest diameter recorded since the treatment started

The appearance of one or more new lesions

In subjects who had no evidence of pleural effusion or ascites at baseline, development of pleural effusion or ascites with cytological confirmation of the neoplastic nature of the effusion/ascites.

Progressive serial elevation of serum CA-125 according to the criteria in Table 1. For progression on the basis of elevated serum CA-125, the date of progression will be the date of the final measurement that meets the criteria in Table 1.

TABLE 1

| | CA-125 | LOWEST CA-125 | CRITERIA TO DOCUMENT |
|---|---|---|---|
| CATEGORY | PRIOR TO THERAPY | DURING THERAPY | PROGRESSION* |
| I | Elevated | Normalized | At least 2x ULN |
| II | Elevated | Not normalized | At least a doubling of the |
| | | | lowest observed value |
| III | Normal | (NA) | At least 2x ULN |

*Elevated values must be confirmed by two separate measurements obtained at lease one week apart

By the term “administering” is meant parenteral, intraperitoneal or oral administration. By “parenteral” is meant intravenous, subcutaneous and intramuscular administration.

The invention further includes a kit for the treatment of cancer patients comprising a vial of a platinum coordination compound, a vial of a taxane and a vial of IFN-γ at doses suitable for use in the methods of the invention as provided in this application. Such kits may contain a label or other suitable instructions for use of the components in the methods of the invention. In a particular aspect, the invention provides a kit for the treatment of ovarian cancer comprising a vial of a platinum coordination compound, a vial of a taxane and a vial of IFN-γ at doses suitable for use in the methods of the invention. One embodiment of this aspect comprises a kit comprising a vial of carboplatin, a vial of paclitaxel and a vial of IFN-γ 1b. Alternatively, the kits of the present invention can comprise one or more vials of IFN-γ, particularly IFN-γ 1b, at doses suitable for use in the methods of the invention as provided in this application together with a label or otherwise suitable instruction for use in combination with chemotherapy using a taxane and a platinum cooordination compound for treatment of cancer, particularly ovarian cancer. In a particular aspect, the invention provides a kit comprising one or more vials of IFN-γ 1b formulated in 20 mg mannitol, 0.36 mg sodium succinate and 0.05 mg polysorbate 20 per 0.1 mg of IFN-γ-1b in sterile water and instructions for administering the IFN-γ-1b to treat ovarian cancer in combination with chemotherapy using paclitaxel and carboplatin at a dose of at least about 0.1 mg at a frequency of at least about three times a week every week during the chemotherapeutic treatment and for at least about three weeks following the completion of the chemotherapeutic treatment.

The following examples are provided by way of illustration and not by way of limitation on the invention.

### EXAMPLE

### Interferon-γ-1b in Combination with Carboplatin and Paclitaxel for Treatment of Advanced Ovarian Cancer

A randomized, double-blind, placebo-controlled, multi-center Phase III trial will evaluate the safety and efficacy of IFN-γ1b in combination with carboplatin and paclitaxel for the first-line therapy of advanced ovarian cancer. The primary endpoint is overall survival, but progression-free survival may be used as a surrogate endpoint.

Patient Population

The patient population for the study will be patients with ovarian or primary peritoneal carcinoma (PPC), FIGO Stage III or IV, who are candidates for first-line chemotherapy under currently used criteria. Generally, patients who have had prior surgery for ovarian cancer or PPC other than primary surgical debulking or those who have had prior malignancies within the past five years other than basal cell or squamous cell carcinomas or in situ carcinoma of the cervix will be excluded from the study.

Randomization

Patients will be randomized in a 1:1 ratio, into two groups: (i) chemotherapy plus IFN-γ1b and (ii) chemotherapy alone. Randomization will be stratified by extent of residual disease and intent to perform interval debulking surgery.

Treatment Plan

Patients will be randomized to receive either chemotherapy plus IFN-γ1b or chemotherapy plus Placebo. Chemotherapy will be paclitaxel (175 mg/m²) over 3 hours, followed on the same day by carboplatin (AUC of 6). The chemotherapy regime will be repeated every 3 weeks. IFN-γ1b (0.1 mg) will be administered s.c. 3 times a week (for example Mon, Wed, Fri) continuously while patients are treated with carboplatin/paclitaxel beginning on the first day of chemotherapy and continuing for three weeks following the last dose of chemotherapy. A total of 6 cycles of chemotherapy will be given unless disease progression or limiting toxicity occurs or patients refuse further treatment.

Progression Assessment

The evidence for progression is implemented as a dichotomous outcome of “alive and without progression at 18 months” with a full disease assessment scheduled at 18 months for those who have not had previously documented disease progression.

The 18-month disease assessment will consist of CT or MRI of the Chest/Abdomen/Pelvis. Prior to 18 months the clinical investigator is to assess disease using usual methodology.

After patients develop objective disease progression or are evaluated at Month 18, further protocol follow-up will be limited to treatment-related adverse events and survival. All patients will be followed until the end of the study.

Criteria for Evaluation

Measurable disease will be defined using the objective RECIST guidelines (Therasse et al. Journal of the National Cancer Institute 2000 92:205). Measurable disease is the presence of at least one lesion that can be accurately measured in at least one dimension (longest dimension to be recorded). Each lesion must be at least 20 mm when measured be conventional techniques (including palpation, plain X-ray, CT and MRI) or at least 10 mm when measured by spiral CT.

Analysis Endpoints

The primary endpoint is overall survival which is the time from randomization until date of death or date last known to be alive. Secondary endpoints will also be measured including progression-free survival, incidence of grade 3 or greater adverse events (based on NCI Common Toxicity Criteria), treatment-failure-free survival and or quality of life. Progression-free survival is the time from randomization until date of diagnosis of progression or date of death without progression.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A method of treating cancer in a patient afflicted therewith which comprises:
(i) administering to such patient a chemotherapeutic treatment comprising an effective amount of a platinum coordination compound and a taxane; and
(ii) administering to such patient at least about 0.1 mg of IFN-γ, at a frequency of at least about three times per week, every week during said chemotherapeutic treatment and for at least about three weeks after the completion of said chemotherapeutic treatment.

2. The method of claim 1, wherein said cancer is ovarian cancer.

3. The method according to claim 2, wherein the platinum coordination compound is selected from the group consisting of carboplatin and cisplatin.

4. The method according to claim 2, wherein the taxane is selected from the group consisting of paclitaxel, docetaxel, and deoxygenated paclitaxel.

5. The method according to claim 2, wherein the IFN-γ is IFN-γ-1b.

6. The method according to claim 2, wherein the platinum coordination compound is carboplatin, the taxane is paclitaxel and the IFN-γ is IFN-γ-1b.

7. The method according to claim 5, wherein the IFN-γ-1b is administered subcutaneously three times per week at a dose of 0.1 mg during the course of treatment with the platinum coordination compound and the taxane, and for three weeks following the final administration of the platinum coordination compound and the taxane.

8. The method of claim 6, wherein the paclitaxel is administered intravenously at a dose of from about 135 mg/m² to about 175 mg/m², the carboplatin is administered intravenously at an AUC of 5 to an AUC of 7, and the IFN-γ1b is administered subcutaneously at about 0.1 mg to about 0.2 mg.

9. The method of claim 8, wherein the paclitaxel and the carboplatin are administered on the same day and repeated every three weeks for up to six times.

10. The method of claim 9, wherein the IFN-γ-1b is administered subcutaneously three times per week at a dose of 0.1 mg during the course of treatment with the carboplatin and the paclitaxel, and for three weeks following the final administration of carboplatin and paclitaxel.

11. A kit comprising a vial containing IFN-γ-1b formulated in 20 mg mannitol, 0.36 mg sodium succinate and 0.05 mg polysorbate 20 per 0.1 mg of IFN-γ-1b in sterile water, and instructions for administering the IFN-γ-1b to treat ovarian cancer in combination with chemotherapy using paclitaxel and carboplatin by administering at least about 0.1 mg of IFN-γ, at a frequency of at least about three times per week, every week during said chemotherapeutic treatment and for at least about three weeks after the completion of said chemotherapeutic treatment.

12. The kit of claim 11, also comprising a vial of paclitaxel and a vial of carboplatin.
